(19) United States
(12) Patent Application Publication
(10) Pub. No.: US 2024/0252500 A1
(43) Pub. Date: Aug. 1, 2024

(54) Treatment of chronic pruritic dermatoses

(71) Applicant: CymaBay Therapeutics, Inc., Newark, CA (US)

(72) Inventors: Yun-Jung Choi, Fremont, CA (US); Charles A. McWherter, Oakland, CA (US)

(73) Assignee: CymaBay Therapeutics, Inc., Newark, CA (US)

(21) Appl. No.: 18/423,232

(22) Filed: Jan. 25, 2024

Related U.S. Application Data
(60) Provisional application No. 63/441,823, filed on Jan. 29, 2023.

(51) Int. Cl. A61K 31/5375; A61K 9/00; A61K 31/192; A61P 17/04

(52) U.S. Cl. CPC A61K 31/5375; A61K 9/0014; A61K 9/0053; A61K 31/192; A61P 17/04

(57) ABSTRACT

Treatment of chronic pruritic dermatoses by administration of seladelpar or a salt thereof, or by administration of mavodelpar or a salt thereof.

### CROSS-REFERENCE TO RELATED APPLICATION

This application claims the benefit under 35 USC 119(e) of Application No. 63/441,823, filed 29 Jan. 2023 and entitled “Treatment of chronic pruritic dermatoses”, the entire content of which is incorporated into this application by reference.

### FIELD OF THE INVENTION

This invention relates to the treatment of chronic pruritic dermatoses.

### DESCRIPTION OF THE RELATED ART

### Pruritus

Pruritus (itch) is a well-known, frequent, and often distressing symptom (i.e., a self-reported subjective experience) of a number of conditions, such as infections, environmental/allergic exposures, skin disorders, drug reactions, and systemic medical disorders. It may be mild and tolerable, but it may also dramatically reduce quality of life, cause severe sleep deprivation and depressive mood, and may even induce suicidal ideation in sufferers most affected by it.

Pruritus may be numerically assessed in several ways. Two single-dimensional numerical assessment methods are the Visual Analog Scale (VAS), in which the patient is presented with a line with the left end-point labeled as “no itching” and the right end-point labeled as “worst possible itching”, and the Numerical Rating Scale (NRS), in which the patient is presented with a line marked like a ruler, typically from 0 to 10 or 0 to 100. In either method, the patient is asked to mark a place on the line corresponding to their present level of itching. The term VAS is sometimes used also to describe a scale where the line is marked like a ruler in addition to having the end-points labeled. The VAS has been validated for use in clinical trials for measuring pruritus and is recommended by the International Forum for the Study of Itch (Ständer et al., “Pruritus Assessment in Clinical trials: Consensus Recommendations from the International Forum for the Study of Itch (IFSI) Special Interest Group Scoring Itch in Clinical Trials”, Acta Derm. Venereol., vol. 93, pp. 509-514 (2013)). A multidimensional numerical assessment method is the 5-D itch scale (Elman et al., “The 5-D itch scale: a new measure of pruritus”, Br. J. Dermatol., vol. 162(3), pp. 587-593 (2010)), which assesses pruritus over the past two weeks on duration (how long per day), degree (how intense), direction (increasing or decreasing), disability (impact of the itching on life), and distribution (body location), with each dimension scored numerically. In clinical trials, pruritus may be recorded on paper or on an electronic device (an e-diary or similar device).

Cholestatic disorders associated with pruritus—where the pruritus is referred to as cholestatic pruritus—include primary biliary cholangitis (PBC) and primary sclerosing cholangitis (PSC). Mu et al., “Implication of Increased Serum IL-31 for Primary Biliary Cholangitis”, Immunol. Invest., 50(6), 662-670 (2021), examined IL-31 in a group of treatment-naïve patients with PBC, comparing them with patients with chronic hepatitis B and with healthy volunteers. They found an increase in serum IL-31 in the PBC patients (median 20.6 pg/mL) relative to the hepatitis B patients (median 11.3 pg/mL) and the healthy volunteers (median 11.0 pg/mL), and considered that “Serum IL-31 is increased in and may be a useful marker for PBC, in particular, for AMA-negative PBC. Furthermore, it is inversely associated with fibrotic progression of PBC.” Mu et al. noted that “Although the key role of IL-31 in pruritus of skin diseases is well known, we found no association of serum IL-31 levels with pruritus in PBC patients.” However, Xu et al., “IL-31 levels correlate with pruritus in patients with cholestatic and metabolic liver diseases and is farnesoid X receptor responsive in NASH”, Hepatol. (2022), https://doi.org/10.1002/hep.32599, testing the farnesoid X receptor (FXR) agonist cilofexor, contradict Mu et al. and report that “Baseline IL-31 levels in PSC and PBC were positively correlated with Visual Analog Scale for pruritus and 5-D itch scores”, and that cilofexor treatment in NASH increased IL-31, and that IL-31 was higher in NASH patients with grade 2-3 pruritus adverse events than in patients with grade 0-1 pruritus adverse events. They also noted that “In a humanized liver murine model, obeticholic acid increased IL-31 mRNA expression in human hepatocytes and serum levels of human IL-31.” From the prescribing information for obeticholic acid (Intercept Pharmaceuticals' OCALIVA), severe pruritus was reported in 23% of patients in the OCALIVA 10 mg arm, 19% of patients in the OCALIVA titration arm, and 7% of patients in the placebo arm in a 12-month double-blind randomized controlled trial of 216 PBC patients.

### Chronic Pruritic Dermatoses and IL-31

Roh et al., “IL-31 Inhibition as a Therapeutic Approach for the Management of Chronic Pruritic Dermatoses”, Drugs, 81, 895-905 (2021), survey the literature on the pathogenesis of chronic pruritus, the role of IL-31 in itch signaling, and the use of IL-31 antagonism as therapeutic approaches for pruritus. They mention as chronic pruritic dermatoses the conditions atopic dermatitis; prurigo nodularis; pruritic autoimmune diseases such as bullous pemphigoid and dermatitis herpetiformis, systemic lupus erythematosus, and dermatomyositis; and, among other dermatologic diseases, statis dermatitis, scabies, mastocystosis, and cutaneous T-cell lymphoma, where they say up to ⅔ of patients present with pruritus and the severity of the pruritus has been correlated with serum IL-31 level. Lotti et al., “Prurigo nodularis and lichen simplex chronicus”, Dermatol. Ther., 21(1), 42-46 (2008) mention lichen simplex chronicus as “a skin disorder characterized by lichenification of the skin as a result of primary excessive scratching.” Kabashima and Irie, “Interleukin-31 as a Clinical Target for Pruritus Treatment”, Front. Med., 8:638325 (2021), mention as pruritic diseases correlated with IL-31 the conditions atopic dermatitis, prurigo nodularis, psoriasis, cutaneous T-cell lymphoma, bullous pemphigoid, chronic urticaria, dermatitis herpetiformis, allergic contact dermatitis, dermatomyositis, chronic pruritus of unknown origin, and “other dermatologic conditions” such as lichen planus, cutaneous (lichen) amyloidosis, statis dermatitis, scleroderma, and the itch associated with wound healing, though they say that detailed data are lacking for these conditions.

Atopic dermatitis (AD, also known as eczema and atopic eczema), is a long-term type of inflammation of the skin (dermatitis). It results in pruritic (itchy), red, swollen, and cracked skin. While the condition may occur at any age, it typically starts in childhood, with changing severity over the years. In children under one year of age, much of the body may be affected; but as children get older, the areas on the insides of the knees and elbows are most commonly affected. In adults, the hands and feet are most commonly affected. Scratching the affected areas worsens the symptoms, and those affected have an increased risk of skin infections. Many people with atopic dermatitis develop hay fever or asthma. The cause of AD is unknown but believed to involve genetics, immune system dysfunction, environmental exposures, and difficulties with the permeability of the skin. If one identical twin is affected, the other has an 85% chance of having the condition.

Excessive type 2 inflammation underlies the pathophysiology of atopic dermatitis. Disruption of the epidermal barrier is thought to play an integral role in the pathogenesis of AD, by allowing allergens to penetrate the epidermis to deeper layers of the skin; ultimately leading to a dysregulated Th2 inflammatory response, which is thought to lead to the eczematous lesions. The Th2 helper T cells become activated, leading to the release of inflammatory cytokines including IL-4, IL-13, and IL-31, which activate downstream Janus kinase (JAK) pathways, leading to inflammation. AD is also associated with the release of pruritogens in the skin, including the Th2 cytokines IL-4, IL-13, IL-31, histamine, and various neuropeptides. Mechanical stimulation from scratching lesions can also lead to the release of pruritogens contributing to the itch scratch cycle whereby there is increased pruritus or itch after scratching a lesion. Chronic scratching of lesions can cause thickening or lichenification of the skin or prurigo nodularis (generalized nodules that are severely itchy). Lichen simplex chronicus (LSC) is thick leathery skin with exaggerated skin markings caused by sudden itching and excessive rubbing and scratching. It generally results in small bumps, patches, scratch marks and scale. People burdened with LSC report pruritus, followed by uncontrollable scratching of the same body region, excessively. Most common sites of LSC are the sides of the neck, the scalp, ankles, vulva, pubis, scrotum, and extensor sides of the forearms. LSC is also associated with atopy, or atopic dermatitis (eczema) and an increase of histamine levels. See, for example, the Wikipedia article “Atopic Dermatitis”, https://en.wikipedia.org/wiki/Atopic_dermatitis, and references cited therein, and the National Institute of Arthritis and Musculoskeletal and Skin Diseases (NIAMS) Health Topics article “Atopic Dermatitis”, https://www.niams.nih.gov/health-topics/atopic-dermatitis, for popular discussions of AD; and Ständer, “Atopic Dermatitis”, N. Engl. J. Med., 384(12), 1136-1143 (2021) for a professional discussion of AD; the Wikipedia articles “Prurigo nodularis”, https://en.wikipedia.org/wiki/Prurigo_nodularis, and “Lichen simplex chronicus”, https://en.wikipedia.org/wiki/Lichen_simplex_chronicus, for popular discussions of these conditions. Rinaldi, “The Itch-Scratch Cycle: A Review of the Mechanisms”, Dermatol. Pract. Concept., 9(2), 90-97 (2019), reviews the originals of itch, the scratch response to itch, and some treatments for pruritic conditions.

Kunimura et al., “The molecular basis for IL-31 production and IL-31-mediated itch transmission: from biology to drug development”, Int'l Immunol., 33(12), 731-736 (2021), say “Atopic dermatitis (AD) is one of the most prevalent chronic inflammatory skin diseases in the world. It is characterized by recurrent eczematous lesions and intense itch, and many cytokines are involved in the pathogenesis of AD.” They note that interleukin-31 (IL-31) has been associated with AD and that serum levels of IL-31 have been correlated with disease severity in AD patients; and that mice treated with intradermal injections of IL-31 and transgenic mice overexpressing IL-31 exhibit scratching behavior and develop severe dermatitis. Kabashima and Irie report that “In clinical studies, levels of serum IL-31 have been found to be elevated in patients with atopic dermatitis, compared with healthy individuals, and decreased after cyclosporin treatment; furthermore, IL-31 levels were shown to correlate with disease severity and pruritic symptoms.” Kabashima et al., “Trial of Nemolizumab and Topical Agents for Atopic Dermatitis with Pruritus”, New Engl. J. Med., 383(2), 141-150 (2020), report a phase 3 study of nemolizumab, a monoclonal antibody that blocks the IL-31 receptor A, in AD; and Ständer et al., “Trial of Nemolizumab in Moderate-to-Severe Prurigo Nodularis”, New Engl. J. Med., 382(8), 706-716 (2020), report a study of nemolizumab in prurigo nodularis.

### Treatments for Chronic Pruritic Dermatoses

Topical corticosteroids, such as hydrocortisone, have proven effective in managing many cases of AD. If topical corticosteroids and moisturizers fail, topical calcineurin inhibitors such as tacrolimus are also used. In 2016, crisaborole (EUCRISA), a PDE-4 inhibitor, was approved as a topical treatment for mild-to-moderate eczema. Other medications used for AD include systemic immunosuppressants such as ciclosporin, methotrexate, interferon γ-1b, mycophenolate mofetil, and azathioprine. Antidepressants and naltrexone may be used to control pruritus. In 2017, dupilumab (DUPIXENT), an IL-4Rα antagonist monoclonal antibody, was approved to treat moderate-to-severe eczema; and tralokinumab (ADBRY), an IL-13 antagonist monoclonal antibody was approved in 2021 for similarly severe cases. Another IL-13 antagonist monoclonal antibody, lebrikizumab, is in phase 3 trials. Two orally-administered JAK inhibitors, abrocitinib (CIBINQUO) and upadacitinib (RINVOQ) have been approved in the US for the treatment of moderate-to-severe eczema. Another JAK inhibitor, ruxolitinib (OPZELURA), is approved as a topical treatment for short-term and non-continuous chronic treatment of mild to moderate eczema (atopic dermatitis) in non-immunocompromised adults and children 12 years of age and older whose disease is not well controlled with topical prescription therapies or when those therapies are not recommended. And, as mentioned above, nemolizumab, an IL-31RA antagonist, is under study in AD and prurigo nodularis. However, the interleukin antagonist antibodies and JAK inhibitors all carry significant risk of side effects, predominantly the risk of opportunistic infections.

### Seladelpar

Seladelpar (International Nonproprietary Name—INN) has the chemical name [4-({(2R)-2-ethoxy-3-[4-(trifluoromethyl)phenoxy]propyl}sulfanyl)-2-methylphenoxy]acetic acid [IUPAC name from WHO Recommended INN: List 77], and the code number MBX-8025. Seladelpar and its synthesis, formulation, and use are disclosed in, for example, U.S. Pat. No. 7,301,050 (compound 15 in Table 1, Example M, claim 49), U.S. Pat. No. 7,635,718 (compound 15 in Table 1, Example M), and U.S. Pat. No. 8,106,095 (compound 15 in Table 1, Example M, claim 14). Lysine (L-lysine) salts of seladelpar and related compounds are disclosed in U.S. Pat. No. 7,709,682 (seladelpar L-lysine salt throughout the Examples, crystalline forms claimed).

Seladelpar is an orally active, potent (2 nM) agonist of peroxisome proliferator-activated receptor-δ (PPARδ). It is specific (>600-fold and >2500-fold compared with peroxisome proliferator-activated receptor-α and peroxisome proliferator-activated receptor-γ receptors). PPARδ activation stimulates fatty acid oxidation and utilization, improves plasma lipid and lipoprotein metabolism, glucose utilization, and mitochondrial respiration, and preserves stem cell homeostasis. According to U.S. Pat. No. 7,301,050, PPARδ agonists, such as seladelpar, are suggested to treat PPARδ-mediated conditions, including “diabetes, cardiovascular diseases, Metabolic X syndrome, hypercholesterolemia, hypo-high density lipoprotein (HDL)-cholesterolemia, hyper-low density protein (LDL)-cholesterolemia, dyslipidemia, atherosclerosis, and obesity”, with dyslipidemia said to include hypertriglyceridemia and mixed hyperlipidemia. Seladelpar, as the L-lysine dihydrate salt, has been studied at oral doses equivalent to 50 and 100 mg/day of seladelpar in mixed dyslipidemia; at doses equivalent to 10, 20, and 50 mg/day of seladelpar in non-alcoholic steatohepatitis; and at doses equivalent to 2, 5, 10, 50, and 200 mg/day of seladelpar in PBC.

US Application Publication No. 2019/0105291 and PCT International Publication No. WO 2019/067373 disclose the treatment of cholestatic pruritus with seladelpar and its salts.

### Mavodelpar

Mavodelpar (INN) has the chemical name (4-{[(2E)-3-(4-fluorophenyl)-3-{4-[3-(morpholin-4-yl)prop-1-yn-1-yl]phenyl}prop-2-en-1-yl]oxy}-2-methylphenoxy)acetic acid [IUPAC name from WHO Recommended INN: List 127], and the code numbers HPP593 and REN001. Mavodelpar and its salts, and its synthesis, formulation, and use are disclosed in, for example, U.S. Pat. No. 7,943,613 (Example 10; claims 1-5); and a synthesis is disclosed in, for example, US Application Publication No. 2023/0416210. The crystalline sodium salt of mavodelpar is disclosed in, for example, U.S. patent Ser. No. 11/267,795. Mavodelpar has an EC₅₀ for PPARδ of 31 nM, while the EC₅₀s for PPARα and PPARγ are over 10 μM, according to the Alzheimer's Drug Discovery Foundation's Cognitive Vitality Report on PPARδ agonists from November 2021. In 2017, mavodelpar was described as a functionally selective PPARδ agonist that has demonstrated a lowering of low-density lipoprotein cholesterol and triglycerides in animal models and humans, with a significant increase in high-density lipoprotein cholesterol. It is also reported to have demonstrated an anti-diabetic effect in several animal models of type 2 diabetes, and that early clinical studies have shown it to be well tolerated. It has been granted Orphan Drug Designation by the FDA for primary mitochondrial myopathies. Most recently, mavodelpar was studied in the STRIDE study (NCT04535609): a global, randomized, double-blind, placebo-controlled pivotal Phase 2b trial of mavodelpar in adult patients with primary mitochondrial myopathies due to mitochondrial DNA defects.

The disclosures of the documents referred to in this application are incorporated into this application by reference.

### SUMMARY OF THE INVENTION

This invention is the treatment of chronic pruritic dermatoses by administration of seladelpar or a salt thereof.

When administered orally for the treatment of primary biliary cholangitis (PBC), seladelpar has been demonstrated to be effective in the treatment of PBC at oral doses of 5, 10, 50, and 200 mg/day, and has been found not to exacerbate cholestatic pruritus; and is expected to be effective in dosages between 0.5 mg/day and 25 mg/day. In studies in PBC at oral doses of 5 and 10 mg/day, seladelpar has been demonstrated to be effective in the treatment of cholestatic pruritus associated with the PBC; and seladelpar has also been shown to reduce IL-31. Because of this reduction of IL-31, seladelpar is also expected to be useful in the treatment of chronic pruritic dermatoses.

This invention is also the treatment of chronic pruritic dermatoses by administration of mavodelpar or a salt thereof. Because mavodelpar is a selective PPARδ agonist, it is expected to reduce IL-31 and therefore be useful in the treatment of chronic pruritic dermatoses.

Preferred embodiments of this invention are characterized by the specification and by the features of Claims 1 to 17 of this application as filed.

### DETAILED DESCRIPTION OF THE INVENTION

### Definitions

Chronic pruritic dermatoses are described in the subsections entitled “Chronic pruritic dermatoses and IL-31”, and “Treatments for chronic pruritic dermatoses” of the DESCRIPTION OF THE RELATED ART. Unless the context requires otherwise, reference to a chronic pruritic dermatosis, and reference to “chronic pruritic dermatoses”, is a reference to any of the conditions described in the “Chronic pruritic dermatoses and IL-31” subsection, and so includes reference to, e.g., atopic dermatitis, prurigo nodularis, lichen simplex chronicus, psoriasis, bullous pemphigoid, dermatitis herpetiformis, systemic lupus erythematosus, dermatomyositis, statis dermatitis, scabies, mastocystosis, cutaneous T-cell lymphoma, chronic urticaria, allergic contact dermatitis, dermatomyositis, chronic pruritus of unknown origin, lichen planus, cutaneous (lichen) amyloidosis, scleroderma, or the itch associated with wound healing.

“Treating” or “treatment” of a chronic pruritic dermatosis in a human includes one or more of:
(1) preventing or reducing the risk of developing pruritus, i.e., causing the pruritus associated with the chronic pruritic dermatosis not to develop in a subject who may be predisposed to the condition for which pruritus is a symptom but who does not yet experience or display the pruritus (i.e. prophylaxis);
(2) inhibiting the pruritus, i.e., arresting or reducing the development of the pruritus; and
(3) relieving the pruritus, i.e., reducing the number, frequency, duration or severity of the pruritus. When the chronic pruritic dermatosis is one caused by an external factor, e.g., the presence of scabies lice in scabies or the presence of lymphoma in cutaneous T-cell lymphoma, it will be understood that the treatment with seladelpar is a treatment for the pruritus and not the external factor.

A “therapeutically effective amount” of seladelpar or a salt thereof means that amount which, when administered to a human for treating a chronic pruritic dermatosis, is sufficient to effect treatment for the pruritus. The therapeutically effective amount for a particular subject varies depending upon the age, health and physical condition of the subject to be treated, the pruritus and its extent, the route of administration of the seladelpar or a salt thereof, the assessment of the medical situation, and other relevant factors. It is expected that the therapeutically effective amount will fall in a relatively broad range that can be determined through routine trial.

Seladelpar is described in the subsection entitled “Seladelpar” of the DESCRIPTION OF THE RELATED ART.

Salts (for example, pharmaceutically acceptable salts) of seladelpar are included in this invention and are useful in the methods described in this application. These salts are preferably formed with pharmaceutically acceptable acids. See, for example, “Handbook of Pharmaceutically Acceptable Salts”, Stahl and Wermuth, eds., Verlag Helvetica Chimica Acta, Zurich, Switzerland, for an extensive discussion of pharmaceutical salts, their selection, preparation, and use. Unless the context requires otherwise, reference to seladelpar, and reference to “a compound that is seladelpar or a salt thereof”, is a reference both to seladelpar itself and to its salts. An amount of a seladelpar salt that is “equivalent to” a particular amount of seladelpar refers to that amount of the salt that is the particular amount multiplied by the ratio of the formula weight of the salt to the formula weight of seladelpar. For example, if seladelpar L-lysine dihydrate salt is being used, since the formula weight of seladelpar L-lysine dihydrate salt is about 1.41 times the formula weight of seladelpar, an amount of about 14.1 mg/day of seladelpar L-lysine dihydrate salt will be equivalent to an amount of 10 mg/day of seladelpar.

Because seladelpar contains a carboxyl group, it may form salts when the acidic proton present reacts with inorganic or organic bases. Typically, seladelpar is treated with an excess of an alkaline reagent, such as hydroxide, carbonate or alkoxide, containing an appropriate cation. Cations such as Na⁺, K⁺, Ca²⁺, Mg²⁺, and NH₄⁺ are examples of cations present in pharmaceutically acceptable salts. Suitable inorganic bases, therefore, include calcium hydroxide, potassium hydroxide, sodium carbonate and sodium hydroxide. Salts may also be prepared using organic bases, such as salts of primary, secondary and tertiary amines, substituted amines including naturally-occurring substituted amines, and cyclic amines, including isopropylamine, trimethylamine, diethylamine, triethylamine, tripropylamine, ethanolamine, 2-dimethylaminoethanol, tromethamine, lysine, arginine, histidine, caffeine, procaine, hydrabamine, choline, betaine, ethylenediamine, glucosamine, N-alkylglucamines, theobromine, purines, piperazine, piperidine, N-ethylpiperidine, and the like. As noted in the “Seladelpar” subsection, seladelpar is currently formulated as its L-lysine dihydrate salt for oral administration.

Mavodelpar is described in the subsection entitled “Mavodelpar” of the DESCRIPTION OF THE RELATED ART. Unless the context requires otherwise, reference to mavodelpar, and reference to “a compound that is mavodelpar or a salt thereof”, is a reference both to mavodelpar itself and to its salts. A “therapeutically effective amount” of mavodelpar or a salt thereof has the same meaning as for seladelpar.

“Comprising” or “containing” and their grammatical variants are words of inclusion and not of limitation and mean to specify the presence of stated components, groups, steps, and the like but not to exclude the presence or addition of other components, groups, steps, and the like. Thus “comprising” does not mean “consisting of”, “consisting substantially of”, or “consisting only of”; and, for example, a formulation “comprising” a compound must contain that compound but also may contain other active ingredients and/or excipients.

### Formulation and Administration

Seladelpar may be administered by any route suitable to the subject being treated and the nature of the subject's condition. Routes of administration include administration by injection, including intravenous, intraperitoneal, intramuscular, and subcutaneous injection, by transmucosal or transdermal delivery, through topical applications, nasal spray, suppository and the like, and oral administration. Formulations may optionally be liposomal formulations, emulsions, formulations designed to administer the drug across mucosal membranes or transdermal formulations. Suitable formulations for each of these methods of administration may be found, for example, in “Remington: The Science and Practice of Pharmacy”, 20th ed., Gennaro, ed., Lippincott Williams & Wilkins, Philadelphia, Pa., U.S.A. When the seladelpar is to be administered systemically, because seladelpar is orally available, typical formulations will be oral, and typical dosage forms will be tablets or capsules for oral administration. As mentioned in the “Seladelpar” subsection, seladelpar has been formulated in capsules for clinical trials. When the seladelpar is to be administered topically, typical formulations will be solutions, suspensions, lotions, ointments, creams, gels, emulsions, foams, and sprays for topical administration. Mayba et al., “A Guide to Topical Vehicle Formulations”, J. Cutan. Med. Surg., 22(2), 207-212 (2018) provide a guide to types of topical formulations and their advantages and disadvantages; while Chang et al., “Generic Development of Topical Dermatologic Products: Formulation Development, Process Development, and Testing of Topical Dermatologic Products”, AAPS J., 15(1), 41-52 (2013), discuss issues related to formulation development, process development, and testing of such products.

Depending on the intended mode of administration, the pharmaceutical compositions may be in the form of solid, semi-solid or liquid dosage forms, preferably in unit dosage form suitable for single administration of a precise dosage if intended for systemic administration. In addition to an effective amount of seladelpar, the compositions may contain suitable pharmaceutically-acceptable excipients, including adjuvants which facilitate processing of the active compounds into preparations which can be used pharmaceutically. “Pharmaceutically acceptable excipient” refers to an excipient or mixture of excipients which does not interfere with the effectiveness of the biological activity of the active compound(s) and which is not toxic or otherwise undesirable to the subject to which it is administered.

For solid compositions, conventional excipients include, for example, pharmaceutical grades of mannitol, lactose, starch, magnesium stearate, sodium saccharin, talc, cellulose, glucose, sucrose, magnesium carbonate, and the like. Liquid pharmacologically administrable compositions can, for example, be prepared by dissolving, dispersing, etc., an active compound as described herein and optional pharmaceutical adjuvants in water or an aqueous excipient, such as, for example, water, saline, aqueous dextrose, and the like, to form a solution or suspension. If desired, the pharmaceutical composition to be administered may also contain minor amounts of nontoxic auxiliary excipients such as wetting or emulsifying agents, pH buffering agents and the like, for example, sodium acetate, sorbitan monolaurate, triethanolamine sodium acetate, triethanolamine oleate, etc.

For oral administration, the composition will generally take the form of a tablet or capsule; or, especially for pediatric use, it may be an aqueous or nonaqueous solution, suspension or syrup. Tablets and capsules are preferred oral administration forms. Tablets and capsules for oral use will generally include one or more commonly used excipients such as lactose and corn starch. Lubricating agents, such as magnesium stearate, are also typically added. When liquid suspensions are used, the active agent may be combined with emulsifying and suspending excipients. If desired, flavoring, coloring and/or sweetening agents may be added as well. Other optional excipients for incorporation into an oral formulation include preservatives, suspending agents, thickening agents, and the like.

A suitable (i.e., a therapeutically effective) amount of seladelpar or a salt thereof for oral administration, when the amount is calculated as seladelpar, is expected to be at least 0.5 mg/day, for example at least 1 mg/day, such as at least 2 mg/day, or at least 5 mg/day; but not more than 200 mg/day, such as not more than 100 mg/day, or not more than 50 mg/day; for example 5 mg/day, 10 mg/day, 25 mg/day, or 50 mg/day for an adult subject with a chronic pruritic dermatosis, depending on the extent and severity of the pruritus and factors such as hepatic and renal function. That is, a suitable amount of seladelpar for oral dosing for adults to treat a chronic pruritic dermatosis is expected to be similar to, but perhaps greater than, the amounts used to treat cholestatic pruritus. These amounts represent an average daily dose, and not necessarily an amount given at a single dose. Dosing may be as frequent as more than once/day (where the amount, or daily dose, will be divided between the number of administrations per day), but will more typically be once/day (where the amount is given in a single administration). Optionally, the dosing may be less frequent than once/day, such as between once/week and every other day, for example once/week, twice/week (especially with the doses at least three days apart), three times/week (especially with the doses at least two days apart), or every other day; so that, as an example, a subject may receive 5 mg twice/week for an amount (daily dose) of 1.4 mg/day. Because of the high oral bioavailability of seladelpar, amounts for intravenous administration are expected to be approximately the same as for oral administration.

For topical administration, the composition will typically comprise, in addition to the seladelpar, fluid or semi-solid vehicles that may include but are not limited to polymers, thickeners, buffers, neutralizers, chelating agents, preservatives, surfactants or emulsifiers, antioxidants, waxes or oils, emollients, sunscreens, and a solvent or mixed solvent system. The solvent or mixed solvent system is important to the formation because it is primarily responsible for dissolving the seladelpar. Optimal solvent or mixed solvent systems are also capable of maintaining clinically relevant levels of the seladelpar in solution despite the addition of a poor solvent to the formulation. The topical compositions may be made into a wide variety of product types, such as lotions, creams, gels, sticks, sprays, ointments, pastes, foams, mousses, and cleansers. These product types can comprise several types of carrier systems, which may include particles, nanoparticles, and liposomes. If desired, disintegrating agents can be added, such as cross-linked polyvinylpyrrolidone, agar, or alginic acid or a salt thereof such as sodium alginate. Techniques for formulation and administration can be found in “Remington: The Science and Practice of Pharmacy”, mentioned above.

Lotions, which are preparations that are to be applied to the skin without friction, are typically liquid or semi-liquid preparations. Lotions will typically contain suspending agents to produce better dispersions as well as compounds useful for localizing and holding the seladelpar in contact with the skin, e.g., methylcellulose, sodium carboxymethylcellulose, or the like. Creams containing the seladelpar for topical administration are viscous liquid or semisolid emulsions, either oil-in-water (O/W) or water-in-oil (W/O). Cream bases are water-washable, and contain an oil phase, an emulsifier and an aqueous phase. The oil phase generally comprises petrolatum or a fatty alcohol, such as cetyl or stearyl alcohol; the aqueous phase usually, although not necessarily, exceeds the oil phase in volume, and generally contains a humectant. The emulsifier in a cream formulation is generally a nonionic, anionic, cationic or amphoteric surfactant. Gel formulations can also be used. Gels are semisolid; and single-phase gels typically contain the active agent (here, seladelpar) distributed substantially uniformly throughout the carrier liquid, which is typically aqueous, but also may be a solvent or solvent blend. Ointments, which are semisolid preparations, are typically based on petrolatum or other petroleum derivatives. The specific ointment base to be used is one that provides for optimum delivery for the seladelpar, and, preferably, additionally provides for other desired characteristics, e.g., emolliency and the like. As with other carriers or vehicles, an ointment base should be inert, stable, nonirritating and non-sensitizing. As explained in “Remington: The Science and Practice of Pharmacy”, ointment bases may be grouped in four classes: oleaginous bases; emulsifiable bases; emulsion bases; and water-soluble bases. Oleaginous ointment bases include, for example, vegetable oils, fats obtained from animals, and semisolid hydrocarbons obtained from petroleum. Emulsifiable ointment bases, also known as absorbent ointment bases, contain little or no water and include, for example, hydroxystearin sulfate, anhydrous lanolin and hydrophilic petrolatum. Emulsion ointment bases are typically either W/O emulsions or O/W emulsions, and include, for example, cetyl alcohol, glyceryl monostearate, lanolin and stearic acid. Preferred water-soluble ointment bases are prepared from polyethylene glycols of varying molecular weight. Useful formulations also include sprays. Sprays generally provide the active agent in an aqueous and/or alcoholic solution which can be misted onto the skin for delivery. Such sprays include those formulated to provide for concentration of the active agent solution at the site of administration following delivery, e.g., the spray solution can be primarily composed of alcohol or similar volatile liquid in which the seladelpar can be dissolved. Upon delivery to the skin, the carrier evaporates, leaving concentrated seladelpar at the site of administration. Topical pharmaceutical compositions may also comprise suitable solid or gel phase carriers. Examples of such carriers include but are not limited to metal salts such as calcium carbonate and calcium phosphate, various sugars, starches, cellulose derivatives, gelatin, and polymers such as polyethylene glycols. The topical pharmaceutical compositions may also comprise a suitable emulsifier, i.e., an agent that enhances or facilitates mixing and suspending O/W or W/O emulsions. The emulsifying agent may consist of a single emulsifying agent or may be a nonionic, anionic, cationic or amphoteric surfactant or blend of two or more such surfactants. Useful emulsifiers include high molecular weight alcohols such as cetearyl alcohol, cetyl alcohol, and stearyl alcohol. Other examples are glyceryl monostearate, ethylene glycol distearate, sorbitan tristearate, propylene glycol monostearate, sorbitan monooleate, sorbitan monostearate, diethylene glycol monolaurate, sorbitan monopalmitate, sucrose dioleate, sucrose stearate, polyoxyethylene lauryl ether, polyoxyethylene (2) stearyl ether, polyoxyethylene (21) stearyl ether, polyoxyethylene monostearate, polyoxyethylene sorbitan monostearate, polyoxyethylene sorbitan monooleate, polyoxyethylene sorbitan monolaurate, and sodium oleate. Emulsifying wax NF (United States National Formulary) is a mixture of cetearyl alcohol and a polysorbate (a polyoxyethylene derivative of a sorbitan fatty acid ester). Cholesterol and cholesterol derivatives may also be employed in externally used emulsions and promote W/O emulsions. Especially suitable nonionic emulsifying agents are those with hydrophile-lipophile balances (HLB) of about 3 to 6 for W/O emulsions and 8 to 18 for O/W emulsions. The topical pharmaceutical compositions may also comprise suitable emollients, which are materials used for the prevention or relief of dryness, as well as for the protection of the skin. Useful emollients include cetyl alcohol, isopropyl myristate, stearyl alcohol, and the like, including mixtures of these materials; and a wide variety of suitable emollients are known to the person of ordinary skill in the art and can be used here.

Topical pharmaceutical compositions may also comprise suitable antioxidants, such as. butylated hydroxytoluene, ascorbic acid, sodium ascorbate, calcium ascorbate, ascorbic palmitate, butylated hydroxyanisole, 2,4,5-trihydroxybutyrophenone, 4-hydroxymethyl-2,6-di-tert-butylphenol, erythorbic acid, gum guaiac, propyl gallate, thiodipropionic acid, dilauryl thiodipropionate, tert-butylhydroquinone, and tocopherols such as vitamin E, and the like, including pharmaceutically acceptable salts and esters of these compounds. They may also comprise suitable preservatives such as benzyl alcohol, methylparaben, propylparaben, chlorobutanol, and the like, and mixtures of these materials; and suitable chelating agents such as ethylene diamine tetraacetic acid (EDTA), ethylene glycol-bis(beta-aminoethyl ether)-N,N,N′,N′-tetraacetic acid (EGTA), and the like. They may also comprise suitable neutralizing agents used to control the pH of the composition to within a pharmaceutically acceptable range; and viscosity increasing agents such as polyacrylic acids. Topical pharmaceutical compositions may also comprise suitable penetration enhancers, to enhance the penetration of the seladelpar through the skin, particularly through the stratum corneum. Penetration enhancers may include water, fatty acids and fatty alcohols, alcohols and glycols, terpenes, sulfoxides, laurocapram, pyrrolidones, and surfactants.

Typically, the seladelpar will be present in a topical formulation in a concentration of from about 0.5% to about 20%; such as 1% to 10%; for example, 1%, 2%, 5%, or 10%. A suitable (i.e., a therapeutically effective) amount of seladelpar or a salt thereof for topical administration will be readily determinable by observation of the change in pruritus on administration of a topical seladelpar formulation.

Typically, a pharmaceutical composition of seladelpar, or a kit comprising compositions of seladelpar, is packaged in a container with a label, or instructions, or both, indicating use of the pharmaceutical composition or kit in the treatment of a chronic pruritic dermatosis.

A person of ordinary skill in the art of the treatment of chronic pruritic dermatoses, who will typically be a physician of ordinary skill in dermatology, will be able to ascertain a therapeutically effective amount of seladelpar or a seladelpar salt for a particular extent of pruritus and patient to achieve a therapeutically effective amount for the treatment of the chronic pruritic dermatosis without undue experimentation and in reliance upon personal knowledge, the skill of the art, and the disclosure of this application.

Mavodelpar may be formulated in a similar manner to seladelpar, and, as with seladelpar, a person of ordinary skill in the art of the treatment of chronic pruritic dermatoses will be able to ascertain a therapeutically effective amount of mavodelpar or a mavodelpar salt for a particular patient and extent of pruritus to achieve a therapeutically effective amount for the treatment of a chronic pruritic dermatosis without undue experimentation and in reliance upon personal knowledge, the skill of the art, and the disclosure of this application.

### EXAMPLES

### Example 1: Seladelpar reduces IL-31 production by CD4 T cells under Th2-promoting conditions

CD4 T cells were enriched from peripheral blood mononuclear cells collected from two normal healthy donors, using the EasySep™ Human CD4+ T Cell Isolation Kit (STEMCELL Technologies). 100,000 CD4 T cells were seeded in a U-bottom 96-well-plate well, and cultured in Th2-promoting conditions promoted by IL-4 or IL-33. From day 1 to day 4, the cells were cultured in the presence of 200 μL T cell culture medium (TCM), 2.5 μL anti-CD3/CD28 beads, 1 μg/mL anti-IL-12/IL-23 antibodies, 50 ng/mL human recombinant IL-4 or 50 ng/mL human recombinant IL-33, and either 10 μM seladelpar in DMSO or DMSO control. On day 5 of culture, the anti-CD3/CD28 beads were diluted by adding 150 μL TCM. From days 5 to 13, the cells were cultured in the presence of the same reagents as for days 1 to 4 plus 50 ng/mL IL-2; and the cell culture medium was renewed every 2-3 days by a change of 50% volume of the medium. On day 13 or day 14, the expanded T cells in an individual well were split to four to six wells of 96-well-plate wells; and 5 ng/mL phorbol myristate acetate and 500 ng/mL ionomycin were added for 3 days to re-activate the T cells. The IL-31 concentration in the culture medium of each well was determined using the V-PLEX Human IL-31 Kit (Meso Scale Diagnostics, LLC; Rockville, MD, USA).

Results are given in the table below, where D represents DMSO control, S represents seladelpar, and SEM is the standard error of the mean; and the numbers are IL-31 concentrations in pg/mL:

| | Donor 1, | | Donor 2, | | Donor 1, | | Donor 2, | |
|---|---|---|---|---|---|---|---|---|
| | IL-4 | | IL-4 | | IL-33 | | IL-33 | |
| | promoted | | promoted | | promoted1 | | promoted | |
| | D | S | D | S | D | S | D | S |
| Mean | 122.18 | 36.00 | 96.61 | 27.28 | 31.48 | 10.14 | 481.78 | 324.05 |
| SEM | 10.57 | 7.78 | 8.16 | 2.88 | 4.21 | 1.82 | 26.11 | 23.98 |

For both donors, and for both promoters, seladelpar reduced IL-31 production by the CD4 T cells under Th2-promoting conditions statistically significantly at p<0.0001, using a non-parametric two-tailed Mann-Whitney test.

### Example 2: Seladelpar does not Increase IL-31 Production in PXB Mice

The PXB mouse (PhoenixBio Co., Ltd., Higashi-Hiroshima, Japan) is a chimeric mouse with a humanized liver, produced by transplanting human hepatocytes into the albumin enhancer/promoter-urokinase-type plasminogen activator complementary DNA transgenic/SCID mouse. The mouse has its liver repopulated by human hepatocytes at a ratio of more than 70%, typically 85-90%, as assessed by measuring the ratio of human albumin to mouse albumin in the blood. The transplanted human hepatocytes express a variety of human messenger RNA (mRNA) and proteins, in a similar manner to those of the normal human liver. The mouse livers consist of human hepatocytes with a small percentage of mouse hepatocytes and mouse hepatic sinusoidal cells (mainly Kupffer cells, endothelial cells, and stellate cells), and the human hepatocytes have been shown to cooperate with mouse hepatic sinusoidal cells in carrying out liver functions.

The study was conducted at PhoenixBio. Male PXB mice aged about 25 weeks, kept in single animal cages and fed CRF1 mouse chow supplemented with AS primate chow for vitamin C supplementation (both from Oriental Yeast Co., Ltd., Tokyo, Japan), were randomized into groups (10/group) based on mean values for body weight and blood human albumin concentration. They were dosed by gavage at 10 mL/kg body weight with vehicle (0.5% carboxymethylcellulose in sterile water), seladelpar (10 mg/mL in vehicle), or obeticholic acid (an FXR agonist, known to cause pruritus in PBC patients, 1 mg/mL in vehicle) once/day for 19 days. Two hours after the last dose, each mouse was anesthetized with isoflurane, at least 400 μL blood was collected by cardiac puncture, and the mouse sacrificed by exsanguination. The blood samples were left to coagulate at room temperature for at least 5 minutes, then centrifuged at 13200×g for 3 minutes at 4° C. to obtain serum, which was analyzed for human IL-31. Relative to day 0 (0.13 pg/mL), both vehicle and seladelpar groups showed only a nominal increase to 0.16 and 0.19 pg/mL, respectively, in serum IL-31 concentration, while obeticholic acid caused a dramatic increase to 3.7 pg/mL.

### Example 3: Seladelpar Lowers IL-31 in PBC (ENHANCE, NCT03602560)

The trial subjects in this phase 3 study were adult, male or female, with a diagnosis of PBC. Exclusion criteria included AST or ALT≥3×ULN, total bilirubin (TBIL)≥2×ULN, autoimmune hepatitis or a history of chronic viral hepatitis, PSC, the current use of fibrates or simvastatin, the use of colchicine, methotrexate, azathioprine, or systemic steroids in the previous two months, the use of an experimental treatment for PBC, and the use of an experimental or unapproved immunosuppressant. Subjects were randomized to receive either placebo, 5 mg/day, or 10 mg/day of seladelpar as the L-lysine dihydrate salt orally in capsule form, once/day dosing, for 12 weeks, with 55, 53, and 53 subjects, respectively, in the three groups. Serum IL-31 and its correlation with patient-reported pruritus numerical rating scale (NRS, 0-10) were assessed.

Baseline IL-31 levels positively correlated with pruritus NRS; r=0.54, p<0.0001). Subjects with NRS≥4 had significantly higher baseline median [interquartile range] IL-31 compared to subjects with pruritus NRS<4 (7.6 pg/mL [1.2, 14.5] vs 1.2 pg/mL [0.3, 2.8], p<0.0001). At baseline, IL-31 was also correlated with serum total bile acids (r=0.54, p<0.0001) and alkaline phosphatase (ALP) (r=0.44, p<0.0001). Seladelpar treatment strongly decreased mean IL-31 levels during the study: placebo (from 4.3 to 3.9 pg/mL, not significant). seladelpar 5 mg/day (from 3.8 to 1.7 pg/mL, p<0.001), and seladelpar 10 mg/day (from 4.2 to 1.7 pg/mL, p<0.001). Subjects with a clinically meaningful improvement in pruritus NRS (≥2 decrease) demonstrated greater dose-dependent reductions in IL-31 from baseline compared to those without pruritus improvement. Significant correlations were also seen between changes in IL-31 vs pruritus NRS (r=0.54, p<0.0001), ALP (r=0.40, p<0.01), and total bile acids (r=0.63, p<0.0001) in the seladelpar 10 mg/day group.

### Example 4: Seladelpar Will Lower the Pruritus of Atopic Dermatitis with Oral Treatment

Adult subjects with pruritic atopic dermatitis are treated orally with doses of 5, 10, 25, and 50 mg/day of seladelpar. Subjects are permitted their usual other medications. The subjects are assessed before the study, and at intervals during the study, such as every 4 weeks during the study and 4 weeks after the last dose of the seladelpar therapy, for safety and pharmacodynamic evaluations. At each visit, the subjects are assessed for their disease symptoms and biomarkers, and assessed for pruritus. The subjects also maintain health diaries, which are reviewed at each visit. The subjects show an improvement in their atopic dermatitis-associated pruritus.

### Example 5: Seladelpar Will Lower the Pruritus of Atopic Dermatitis with Topical Treatment

Adult subjects with pruritic atopic dermatitis are treated topically with an ointment containing 1, 2, 5, or 10% of seladelpar. Subjects are permitted their usual other medications. The subjects are assessed before the study, and at intervals during the study, such as every 4 weeks during the study and 4 weeks after the last dose of the seladelpar therapy, for safety and pharmacodynamic evaluations. At each visit, the subjects are assessed for their disease symptoms and biomarkers, and assessed for pruritus. The subjects also maintain health diaries, which are reviewed at each visit. The subjects show an improvement in their atopic dermatitis-associated pruritus.

### Example 6: Mavodelpar Will Lower the Pruritus of Atopic Dermatitis with Oral Treatment

Adult subjects with pruritic atopic dermatitis are treated orally with doses of 5, 10, 25, 50, and 100 mg/day of mavodelpar. Subjects are permitted their usual other medications. The subjects are assessed before the study, and at intervals during the study, such as every 4 weeks during the study and 4 weeks after the last dose of the mavodelpar therapy, for safety and pharmacodynamic evaluations. At each visit, the subjects are assessed for their disease symptoms and biomarkers, and assessed for atopic dermatitis-associated pruritus. The subjects also maintain health diaries, which are reviewed at each visit. The subjects show an improvement in their atopic dermatitis-associated pruritus.

### Example 7: Mavodelpar Will Lower the Pruritus of Atopic Dermatitis with Topical Treatment

Adult subjects with pruritic atopic dermatitis are treated topically with an ointment containing 1, 2, 5, or 10% of mavodelpar. Subjects are permitted their usual other medications. The subjects are assessed before the study, and at intervals during the study, such as every 4 weeks during the study and 4 weeks after the last dose of the mavodelpar therapy, for safety and pharmacodynamic evaluations. At each visit, the subjects are assessed for their disease symptoms and biomarkers, and assessed for pruritus. The subjects also maintain health diaries, which are reviewed at each visit. The subjects show an improvement in their atopic dermatitis-associated pruritus.

While this invention has been described in conjunction with specific embodiments and examples, it will be apparent to a person of ordinary skill in the art, having regard to that skill and this disclosure, that equivalents of the specifically disclosed materials and methods will also be applicable to this invention; and such equivalents are intended to be included within the following claims.

1. A method of treating a chronic pruritic dermatosis in a subject by administration to that subject of a therapeutically effective amount of a compound that is seladelpar or a salt thereof.

2. The method of claim 1 where the compound is a seladelpar L-lysine salt.

3. The method of claim 2 where the compound is seladelpar L-lysine dihydrate salt.

4. The method of claim 1 where the administration is oral administration.

5. The method of claim 1 where the amount of the compound is equivalent to between 0.5 mg/day and 200 mg/day of seladelpar.

6. The method of claim 5 where the amount of the compound is equivalent to at least 1 mg/day of seladelpar.

7. The method of claim 5 where the amount of the compound is equivalent to not more than 100 mg/day of seladelpar.

8. The method of claim 7 where the amount of the compound is equivalent to 5 mg/day, 10 mg/day, 25 mg/day, or 50 mg/day of seladelpar.

9. The method of claim 1 where the administration is topical administration.

10. The method of claim 1 where the administration is once/day.

11. The method of claim 1 where the administration is more than once/day.

12. The method of claim 1 where the administration is between once/week and every other day.

13. The method of claim 1 where the chronic pruritic dermatosis is atopic dermatitis, prurigo nodularis, lichen simplex chronicus, psoriasis, bullous pemphigoid, dermatitis herpetiformis, systemic lupus erythematosus, dermatomyositis, statis dermatitis, scabies, mastocystosis, cutaneous T-cell lymphoma, chronic urticaria, allergic contact dermatitis, dermatomyositis, chronic pruritus of unknown origin, lichen planus, cutaneous (lichen) amyloidosis, scleroderma, or the itch associated with wound healing.

14. A method of treating a chronic pruritic dermatosis in a subject by administration to the subject of a therapeutically effective amount of a compound that is mavodelpar or a salt thereof.

15. The method of claim 14 where the compound is mavodelpar sodium salt.

16. The method of claim 14 where the administration is oral administration.

17. The method of claim 14 where the administration is topical administration.

18. The method of claim 14 where the chronic pruritic dermatosis is atopic dermatitis, prurigo nodularis, lichen simplex chronicus, psoriasis, bullous pemphigoid, dermatitis herpetiformis, systemic lupus erythematosus, dermatomyositis, statis dermatitis, scabies, mastocystosis, cutaneous T-cell lymphoma, chronic urticaria, allergic contact dermatitis, dermatomyositis, chronic pruritus of unknown origin, lichen planus, cutaneous (lichen) amyloidosis, scleroderma, or the itch associated with wound healing.
